(19) United States
(12) Patent Application Publication
(10) Pub. No.: US 2016/0206767 A1
(43) Pub. Date: Jul. 21, 2016

(54) STERILIZING APPARATUS AND STERILIZING METHOD USING WET GASEOUS CHLORINE DIOXIDE

(71) Applicant: OXY THERAPY, INC., Ansan-si, Gyeonggi-do (KR)

(72) Inventors: Kyoung Ho PARK, Seoul (KR); Kyeong Hwan PARK, Suwon-si (KR); Min Gyu HWANG, Seongnam-si (KR); Keoung Hwa LEE, Incheon (KR)

(21) Appl. No.: 14/914,315

(22) PCT Filed: Aug. 7, 2014
(86) PCT No.: PCT/KR2014/007322

§ 371 Date: Feb. 25, 2016

(30) Foreign Application Priority Data
Aug. 26, 2013 (KR) 10-2013-0100798

(51) Int. Cl. A61L 2/20

(52) U.S. Cl. CPC A61L 2/20

(57) ABSTRACT

The present invention provides a sterilizing apparatus using wet gaseous chlorine dioxide, the sterilizing apparatus comprising: a chamber provided with a sterilization chamber; a gaseous chlorine dioxide generating part for generating wet gaseous chlorine dioxide from stabilized chlorine dioxide; a photoionization detecting part for measuring the concentration of the gaseous chlorine dioxide in the chamber; an electronic control part for controlling a sterilization process; a gaseous chlorine dioxide adsorption removal part for removing gaseous chlorine dioxide remaining in the chamber after the sterilization process has been completed; an operation panel consisting of an input portion for inputting sterilization conditions and a display portion for displaying a sterilization state; and a printer for outputting a sterilization history. In addition, the present invention provides a sterilizing method using wet gaseous chlorine dioxide in the sterilizing apparatus, the sterilizing method characterized by: generating, in the gaseous chlorine dioxide generating part, wet gaseous chlorine dioxide having a humidity of 65%˜95% by irradiating stabilized chlorine dioxide with ultraviolet light having a maximum peak wavelength of 253.7 nm and emission output of 1.2˜11 mW/cm²; and sterilizing even spore by exposing an object being sterilized to the wet gaseous chlorine dioxide for a certain amount of time.

### TECHNICAL FIELD

The present invention relates to a sterilizing apparatus and method, and in particular to a sterilizing apparatus and method using a wet gaseous chlorine dioxide wherein a sterilization object, for example, a medical or surgical operation instrument, etc. is inputted in a sterilization chamber of a sterilization apparatus, and a wet gaseous chlorine dioxide including a predetermined level of humidity is generated using a stabilized gaseous chlorine dioxide under an environment of a room temperature and a normal pressure, and microorganism, for example, bacteria or fungus, included in a sterilization object can be removed in an easy and fast way.

### BACKGROUND ART

The method for sterilizing microorganism in general is classified into six methods, for example, a high pressure steam method, a dry heat method, a radiation method, a high frequency method, a gas method, and a filter method, among which the gas method is a method wherein microorganism is sterilized and become extinct using a sterilization gas. The sterilization gas may be any of ethylene oxide (C₂H₄O), formaldehyde (HCHO), hydrogen peroxide (H₂O₂), and chlorine dioxide (ClO₂).

These gases may have different temperature, humidity, gas concentration, sterilization time, etc. during sterilization depending on the kinds of gas, and some of the gases may have a bad effect on a human body. For this reason, a careful attention should be paid on use environment and residual gas concentration. According to the gas method, the extinction of microorganism after sterilization cannot be quantitatively measured or estimated.

The gaseous chlorine dioxide designated as a sterilization gas has a boiling point of 11° C. and is a yellowish green gas which exists in a gas state and has a strong oxidizing power. If the gaseous chlorine dioxide accounts for higher than 10% in the air, it may explode. For this reason, it is impossible to store in a container like an ethylene oxide gas, whereupon any application to a gaseous chlorine dioxide sterilization apparatus based on the gas sterilization method has been almost impossible so far.

In recent years, as for chlorine dioxide, a gaseous chlorine dioxide has been commercially available and recognized as a sterilization gas. A research result shows that a sterilization disinfection using a gaseous chlorine dioxide which has an effect on sterilization in a wide range of microorganism, for example, virus, bacteria, fungus, etc. could be used as a measuring means for influenza virus, fruit, vegetables and livestock diseases. So, the gaseous chlorine dioxide is being directly applied to the industry, but the sterilization actually needs a vacuum operation and a heating operation, and the operation and work are inconvenient.

As a prior technology on a sterilization apparatus and method using chlorine dioxide, the Korean patent registration No. 10-1131607 (laid-open on Mar. 30, 2012) recites a “low temperature sterilization and disinfection device using a continuously operable chlorine dioxide and a sterilization method using the same”, wherein a gaseous chlorine dioxide generated in such a way to process NaClO₂ using a steam separation membrane in an electrolysis method is stored in an auxiliary chamber, and the gaseous chlorine dioxide stored in the auxiliary chamber is supplied to a main chamber where a sterilization operation is being actually carried out. This prior technology inevitably entails a complicated process wherein it needs to control and operation seven valves while maintaining a high vacuum state in the main chamber. It does not disclose any method to control constant the concentration of the gaseous chlorine dioxide in the auxiliary chamber and the main chamber. If the sterilization is actually carried out in the above state, the sterilization time cannot be accurately set due to a deviation in the concentration of the gaseous chlorine dioxide, and the input of any appropriate level humidity level in the sterilization chamber and a humidity maintaining are impossible, so it is impossible to sterilize spore which is present in the form of a spore state.

### DISCLOSURE OF INVENTION

Accordingly, the present invention is made in an effort to resolve the above problems. It is an object of the present invention to provide a sterilization apparatus wherein a vacuum operation is not necessary, and a wet gaseous chlorine dioxide of which a predetermined amount of moisture is contained in a gaseous chlorine dioxide can be in real time generated at a room temperature without any pretreatment process to maintain a relative humidity inside a chamber, by means of which even spores in a sterilization object can be sterilized, and a wet gaseous chlorine dioxide can be removed within a short time.

It is another object of the present invention to provide a wet gaseous chlorine dioxide sterilization apparatus wherein a gaseous chlorine dioxide inside a chamber can be charged at a concentration of up to 1,000 ppm to the maximum and can be adjusted, and if a sterilization object is exposed, without any vacuum or pressurization process, to an environment wherein the concentration of a wet gaseous chlorine dioxide is 80˜120 ppm, and an exposure time is 20˜120 minutes, a sterilization can be finished within 130 minutes.

It is further object of the present invention to improve the reliability and efficiency of a sterilization apparatus through a standardization and automation of a sterilization in such a way to automatically adjust and control a wet gaseous chlorine dioxide concentration, a sterilization environment and a sterilization process with the aid of an electronic control system.

It is still further another object of the present invention to provide a wet gaseous chlorine dioxide sterilization apparatus which is able to sterilize, under an environment of a room temperature and a normal pressure, an unpacked medical instrument and a surgical operation instrument, a metallic and polymer-made medical instrument, which is weak to a high temperature, a high pressure and humidity, absorbable and porous medical goods (a cellulose, a woven cloth, paper, boards, and a sponge), and a medical instrument and a surgical instrument wherein a sterilization object is packed with a Tyvek.

### Technical Solution

To achieve the above objects, there is provided a sterilization apparatus using a wet gaseous chlorine dioxide, which may include, but is not limited to, a chamber 2 which includes a sterilization chamber 1; a gaseous chlorine dioxide generating part 3 which is able to generate a wet gaseous chlorine dioxide from a stabilized chlorine dioxide; a photoionization detecting part 4 which is able to measure the concentration of a gaseous chlorine dioxide in the chamber; an electronic control part 5 which is provided to control a sterilization process; a gaseous chlorine dioxide adsorption removal part 6 which is able to remove a gaseous chlorine dioxide remaining in the chamber after a sterilization process has been completed; an operation panel 7 which is formed of an input portion to input a sterilization condition, and a display portion to display a sterilization state; and a printer 8 which is provided to output a sterilization history.

The gaseous chlorine dioxide generating part 3 includes a ultraviolet ray lamp 3a wherein a chlorine dioxide is formed in a melted state, a semi-solid matter state or a solid matter state to generate a wet gaseous chlorine dioxide having a relative humidity of 65%˜95% via a photochemical reaction, and the moisture content thereof is 70%˜98%, and the concentration of the gaseous chlorine dioxide is 0.1%˜15% (1,000 ppm˜150,000 ppm), and the rest is formed of a stabilized gaseous chlorine dioxide formed of a recipient (an agar), and a predetermined amount of the ultraviolet ray is radiated from the ultraviolet ray lamp toward to the stabilized chlorine dioxide, thus generating a wet gaseous chlorine dioxide, and the ultraviolet ray lamp has the maximum peak wavelength of 253.7 nm, and the ultraviolet ray radiation output thereof has a capacity of 1.2˜11 mW/cm².

The photoionization detecting part 4 is provided to detect concentration using a photoionization detecting sensor which is able to ionize and capture the electrons of the gaseous chlorine dioxide among the wet gaseous chlorine dioxide generated in the chamber and transmit the detected measurement values to the electronic control part 5.

The electronic control part 5 is able to convert a sterilization condition set on the operation panel and a concentration measurement value of the wet gaseous chlorine dioxide received from the photoionization detecting sensor into an electrical signal and display the converted electrical signal, and is able to control the concentration of the gaseous chlorine dioxide, and is able to control a door locking device for the door to be opened or closed only when the gaseous chlorine dioxide remaining in the chamber after the sterilization is completed, is completely removed, and is able to display values and graphs generated during the sterilization procedure, on the operation panel, and the sterilization history is printed using the printer, wherein the above control operations are automatically carried out by a control of the electronic control part.

The gaseous chlorine dioxide adsorption removal part 6 includes a filter containing an active carbon with a particle diameter of 0.01˜0.1 μm, a zeolite with a particle diameter of 0.5˜2 mm, and sodium thiosulfate 5H₂O in order for the wet gaseous chlorine dioxide remaining in the chamber after the sterilization process is completed, to be completely removed, and the filter adsorbs and removes the wet gaseous chlorine dioxide in the chamber by circulating it using a filtering fan after the sterilization process is completed.

The wet gaseous chlorine dioxide sterilization apparatus is able to sterilize, under an environment of a room temperature and a normal pressure, a sterilization object, for example, an unpacked medical instrument, metallic and polymer-made medical instruments, which are weak to a high temperature, a high pressure and humidity, absorbable and porous medical goods, and a medical instrument which is sealingly packed using a Tyvek, thus sterilizing spores.

The operation panel 7 is a touch screen panel and is formed of an input portion to input a sterilization condition and a display portion to display a sterilization state, and the input portion may include a start/stop button 7a which is provided to control the start and stop of the sterilization apparatus; a sterilization progress time setting unit 7b which is able to set a sterilization progress time; a wet gaseous chlorine dioxide concentration setting unit 7c which is provided to set an upper limit value and a lower limit value of the wet gaseous chlorine dioxide concentration; and a circulation fan air flow rate adjusting button 7d which is able to adjust the concentration of the wet gaseous chlorine dioxide by adjusting the air flow rate of the circulation fan installed inside of a sterilization chamber, and the display portion may include a door state display unit 7e which is able to display an opening and closing state of the door of the sterilization apparatus; a communication state display unit 7f which is able to display a communication connection state between the operation panel 7 and the electronic control part 5; a medicine remainder display unit 7g which is provided to display the remainder of the medicine (a stabilized chlorine dioxide); an air flow rate display unit 7h which is able to display the air flow rate of the circulation fan inside the sterilization chamber; a wet gaseous chlorine dioxide state display unit 7i which is able to display the temperature, humidity and concentration of the wet gaseous chlorine dioxide; a sterilization time display unit 7j which is provided to display the current time, a sterilization start time and a sterilization completion time; and a graph screen 7k which is configured to display in real time in the form of a graph the temperature, humidity and concentration of the wet gaseous chlorine dioxide based on the driving time of the sterilization apparatus.

The screen displayed on the operation panel 7 is formed of a logo screen, an operation screen, a door opening and closing screen, a medicine remainder display screen, a gas removal screen and an operation completion screen, which are separately displayed based on a progress state of the sterilization.

To achieve the above objects, there is provided a sterilization method using a gaseous chlorine dioxide, which may include, but is not limited to, generating a wet gaseous chlorine dioxide having a humidity of 65%˜95% by radiating to a stabilized chlorine dioxide an ultraviolet ray having the maximum peak wavelength of 253.7 nm and a radiation output of 1.2˜11 mW/cm² in a gaseous chlorine dioxide generating part 3; and sterilizing spores in such a way to expose a sterilization object to the wet gaseous chlorine dioxide for a predetermined time.

The sterilization method using a wet gaseous chlorine dioxide may include, but is not limited to, a sterilization condition setting step (S1) wherein a sterilization time, a gaseous chlorine dioxide concentration, and a circulation fan air flow rate are set at an operation panel of the sterilization apparatus; an operation selection step (S2) wherein an operation button of an operation/stop button provided at the operation panel 7 is selected; a medicine remainder confirmation step (S3) wherein the remainder of a medicine is confirmed at a medicine remainder display unit; a door closing step (S4) wherein a door is closed after the opening and closing state of the door of the sterilization apparatus is checked; a ultraviolet ray lamp ON step (S5) wherein a ultraviolet ray lamp is turned on as soon as the door is closed and locked; a wet gaseous chlorine dioxide circulation step (S6) wherein the wet gaseous chlorine dioxide is circulated in the sterilization chamber by turning on the circulation fan inside the sterilization chamber; a data collection step (S7) wherein the data on a temperature, humidity, concentration and sterilization progress time of the wet gaseous chlorine dioxide are collected; a confirmation step (S8) wherein the sterilization progress time completion is checked; a ultraviolet ray lamp OFF step (S9) wherein the ultraviolet ray lamp is turned off; a filtering fan ON step (S10) wherein a filtering fan is driven to filter the wet gaseous chlorine dioxide; a wet gaseous chlorine dioxide removal step (S11) wherein the circulation fan and the filtering fan are driven until the concentration of the wet gaseous chlorine dioxide becomes 0 (zero) ppm; a door lock releasing step (S12) wherein when the concentration of the wet gaseous chlorine dioxide is 0 ppm, the circulation fan and the filtering fan are stopped, and the door lock state is released; a sterilization progress content printing step (S13) wherein a sterilization progress content is printed using a printer; and a sterilization standby step (S14) wherein a sterilization product is removed from the sterilization chamber, and a standby state is maintained for the sake of the next sterilization.

### Advantageous Effects

The wet gaseous chlorine dioxide sterilization apparatus according to the present invention does not need any vacuum operation, and a wet gaseous chlorine dioxide wherein 65%˜95% of moisture is contained in a gaseous chlorine dioxide can be generated in real time under an environment of a normal pressure and a room temperature without any additional pretreatment process to maintain a relative humidity inside a chamber, thus sterilizing spores in a sterilization object, and a wet gaseous chlorine dioxide can be removed within a short time.

The gaseous chlorine dioxide can be charged in the chamber at a concentration of up to 1,000 ppm to the maximum, and the concentration can be adjusted, and if a sterilization object is exposed to a wet gaseous chlorine dioxide at a concentration of 80˜120 ppm for 20˜120 minutes without any vacuum or pressurization operation, the sterilization can be finished within about 130 minutes, whereupon the sterilization apparatus can be manufactured simplified, and a quick sterilization is available.

Moreover, the wet gaseous chlorine dioxide concentration, sterilization environment and sterilization process can be automatically adjusted and controlled by an electronic control part, thus improving reliability and efficiency through the standardization and automation of the sterilization apparatus.

Furthermore, the door can be opened or closed only if the gaseous chlorine dioxide in the chamber is completely removed after the sterilization is finished, which is able to provide safety to a worker. The whole work processes can be indicated in number and graph for the sake of visible observation, and a result of the sterilization can be printed by a printer, so a sterilization process can be visually checked.

In addition, the present invention is able to sterilize, under an environment of a room temperature and a normal pressure, an unpacked medical instrument and a surgical operation instrument, a metallic and polymer-made medical instrument, which is weak to a high temperature, a high pressure and humidity, absorbable and porous medical goods (a cellulose, a woven cloth, paper, boards, and a sponge), and a medical instrument and a surgical instrument wherein a sterilization object is packed with a tieback.

### BRIEF DESCRIPTION OF DRAWINGS

FIG. 1 is a view illustrating an exterior of a sterilization apparatus using a wet gaseous chlorine dioxide according to the present invention.

FIG. 2 is a concept view illustrating a major configuration of a sterilization apparatus using a wet gaseous chlorine dioxide according to the present invention.

FIG. 3 is a view illustrating an input unit and a display unit which are provided at an operation panel of a sterilization apparatus using a wet gaseous chlorine dioxide according to the present invention.

FIG. 4 is a view illustrating a screen displayed on an operation panel of a sterilization apparatus using a wet gaseous chlorine dioxide according to the present invention.

FIG. 5 is a view illustrating a gaseous chlorine dioxide sterilization procedure using a wet gaseous chlorine dioxide according to the present invention.

FIG. 6 is a graph showing a relative humidity inside a chamber, which changes based on an operation time of a sterilization embodiment of a wet gaseous chlorine dioxide sterilization apparatus according to the present invention.

FIG. 7 is a photo showing the colors of a culture medium used during a sterilization and a sterilized and non-sterilized culture medium in a wet gaseous chlorine dioxide sterilization apparatus according to the present invention.

### BEST MODES FOR CARRYING OUT THE INVENTION

The sterilization apparatus and method using a wet gaseous chlorine dioxide according to the present invention will be described with reference to the accompanying drawings.

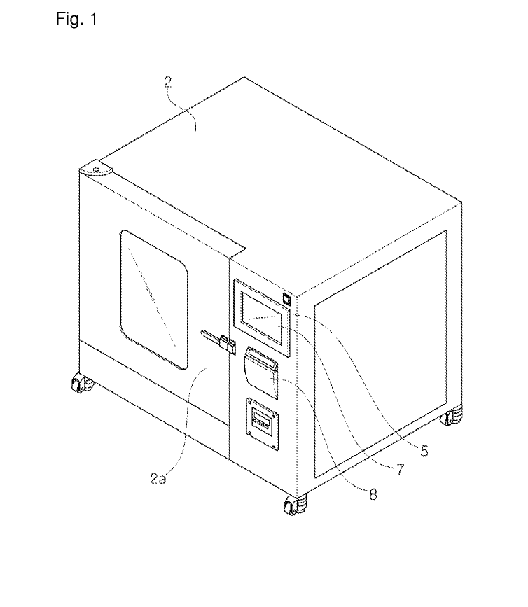

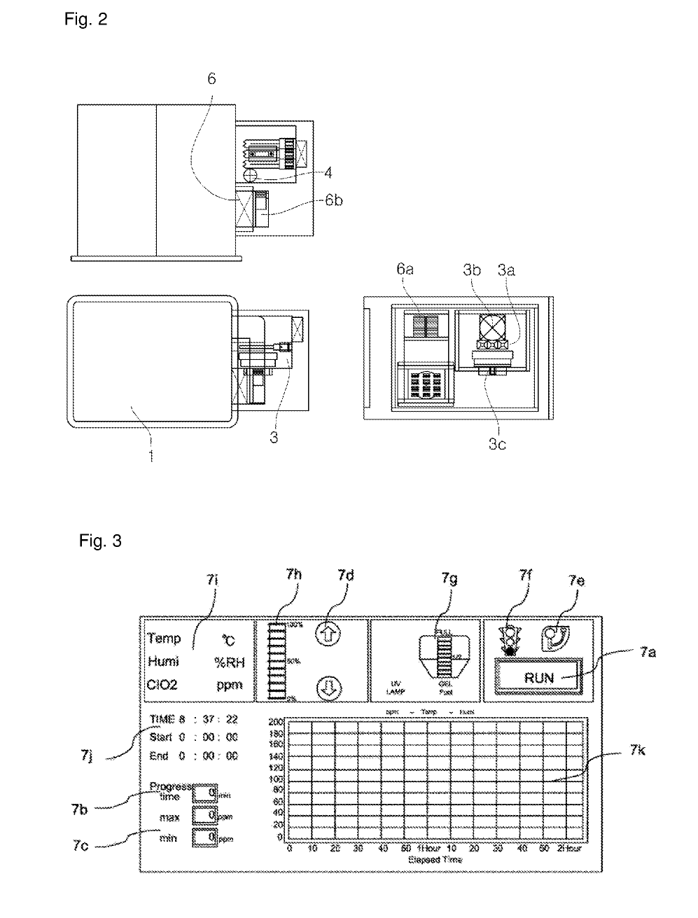

Referring to FIGS. 1 and 2, the sterilization apparatus using a wet gaseous chlorine dioxide may include, but is not limited to, a chamber 2 which includes a sterilization chamber 1; a gaseous chlorine dioxide generating part 3 which is able to generate a wet gaseous chlorine dioxide from a stabilized chlorine dioxide; a photoionization detecting part 4 which is able to measure the concentration of a gaseous chlorine dioxide in the chamber; an electronic control part 5 which is provided to control a sterilization process; a gaseous chlorine dioxide adsorption removal part 6 which is able to remove a gaseous chlorine dioxide remaining in the chamber after a sterilization process has been completed; an operation panel 7 which is formed of an input portion to input a sterilization condition, and a display portion to display a sterilization state; and a printer 8 which is provided to output a sterilization history.

The chamber 2 is configured in a typical experiment instrument type wherein the interior and space of a sterilization chamber and an exterior of a chamber are formed in a hexahedron structure so that a sterilization process can be carried out using a gaseous chlorine dioxide under an environment of a room temperature and a normal pressure unless the interior of the sterilization chamber is heated or pressurized or without forming a special environment, for example, a vacuum state, etc., and the sterilization chamber 1 can be opened or closed by a door 2a, and the inside of the sterilization chamber can be viewed through a transparent window installed at a portion of the door, and the whole components of the chamber 2 can be easily moved with the aid of a caster.

The gaseous chlorine dioxide generating part 3 is an apparatus which is able to generate gaseous chlorine dioxide through a photochemical reaction by radiating a predetermined ultraviolet ray toward chlorine dioxide, wherein a wet gaseous chlorine dioxide can be generated through a photochemical reaction in such a way that an ultraviolet ray lamp 3a radiates a predetermined amount of ultraviolet ray in a state where a stabilized chlorine dioxide (an aqueous solution, a semi-solid matter, and a solid matter) in a separated region, which has a predetermined volume, in the sterilization chamber the space of which is intensively formed without using any partitions, and the generated wet gaseous chlorine dioxide is circulated by a circulation fan 3b.

The chlorine dioxide is formed in a melted state, a semi-solid matter state or a solid matter state to generate a wet gaseous chlorine dioxide having a relative humidity of 65%˜95% for a gaseous chlorine dioxide to sterilize spores, and the moisture content thereof is 70%˜98%, and the concentration of gaseous chlorine dioxide is 0.1%˜15% (1,000 ppm˜150,000 ppm), and the rest is formed of a stabilized gaseous chlorine dioxide which may function as a recipient, for example, an agar, etc. The stabilized chlorine dioxide is placed at a medicine holder 3c provided at a gaseous chlorine dioxide generating part 3 inside the chamber.

If the moisture content of the stabilized chlorine dioxide is less than 70%, the wet gaseous chlorine dioxide generated by the gaseous chlorine dioxide generating part may have a relative humidity of less than 65%, and if the concentration is less than 0.1%, the concentration of the wet gaseous chlorine dioxide in the chamber may not reach over 60 ppm, which means that it is difficult to obtain a range to sterilize spores which are the target of sterilization, whereupon a non-sterilized state may occur. For this reason, it is preferred that the stabilized chlorine dioxide has a moisture content of 70%˜98%, and a concentration of 0.1%˜15% (1,000 ppm˜150,000 ppm).

Since it is preferred that the gaseous chlorine dioxide of the wet gaseous chlorine dioxide has a concentration of 400 ppm˜1000 ppm, a ultraviolet ray lamp 3a may be provided, wherein the ultraviolet ray which is able to cause a photochemical reaction to a stabilized chlorine dioxide has a short wavelength region wherein the maximum peak wavelength is 253.7 nm, and a radiation output is 1.2˜11 mW/cm², by means of which a ultraviolet ray can be radiated to the stabilized chlorine dioxide.

If the ultraviolet ray has a maximum peak wavelength of less than 253.7 nm, and the radiation output is less than 1.2 mW/cm², the concentration of the gaseous chlorine dioxide generated from the stabilized gaseous chlorine dioxide may be less than 400 ppm, and the maximum peak wavelength may be over 253.7 nm. If the radiation output is over 11 mW/cm², the concentration of the gaseous chlorine dioxide may be over 1000 ppm. In this case, the spores which are the target of sterilization may not be sterilized. For this reason, it needs to radiate a ultraviolet ray the maximum peak wavelength of which is 253.7 nm, and the radiation output is 1.2˜11 mW/cm².

The photoionization detecting part 4 may be formed of a photoionization detecting sensor which is able to ionize and capture the electrons of chlorine dioxide among the wet gaseous chlorine dioxide so as to measure the concentration of the gaseous chlorine dioxide among the wet gaseous chlorine dioxide generated in the chamber, and a measured value detected by the photoionization detecting sensor is transmitted to an electronic control part 5.

The electronic control part 5 is a device to control a sterilization process in order for the following procedures to be automatically carried out, wherein a sterilization progress time, the upper and lower limit values of the concentration of a gaseous chlorine dioxide, and a concentration measurement value of a wet gaseous chlorine dioxide received from the photo ionization detection sensor, which are the sterilization process conditions set by a user at the operation panel, are converted into electrical signals, thus indicating a sterilization time, and the concentration of the gaseous chlorine dioxide is controlled, and it is controlled that the door is opened or closed only when the gaseous chlorine dioxide in the chamber is completely removed after the sterilization is finished, and a door locking device provided for the sake of a user's safety is controlled, and it is controlled that the whole matters on the sterilization process during the sterilization are displayed in the form of number and graph on the operation panel 7 in order for the workers to visually check, and the contents on the sterilization are outputted by the printer 8.

The gaseous chlorine dioxide adsorption unit 6 is a gaseous chlorine dioxide adsorption removing unit which is able to remove a wet gaseous chlorine dioxide remaining in the chamber after the sterilization process is finished, and is formed of a filter 6a including an active carbon with a particle diameter of 0.01˜0.1/CM, a zeolite with a particle diameter of 0.5˜2 mm, and sodium thiosulfate 5H₂O. The gaseous chlorine dioxide is adsorbed by the pores of the filter 6a and removed, while continuously circulating the wet gaseous chlorine dioxide in the chamber with the aid of the filter 6b after the sterilization is finished.

If the particle diameter of the active carbon is less than 0.01 μm, and the particle diameter of the zeolite is less than 0.5 mm, the moisture contained in the wet gaseous chlorine dioxide may gather among the particles of the active carbon and the zeolite, for which the portions among the particles may be blocked before the gaseous chlorine dioxide is adsorbed. If the particle diameter of the active carbon is over 0.1 μm, and the particle diameter of the zeolite is over 2 mm, the gaseous chlorine dioxide contained in the wet chlorine dioxide may directly pass through the particles, which means that the adsorption and removal may not occur normally. Given this condition, it is preferred that the particle diameter of the active carbon is 0.01˜0.1 μm, and the particle diameter of the zeolite is 0.5˜2 mm.

The operation panel 7 is a touch screen panel. As illustrated in FIG. 3, it is formed of an input portion where a sterilization condition is inputted, and a display portion where a sterilization state is displayed.

The input portion may include a start/stop button 7a which is provided to control the start and stop of the sterilization apparatus; a sterilization progress time setting unit 7b which is able to set a sterilization progress time; a wet gaseous chlorine dioxide concentration setting unit 7c which is provided to set an upper limit value and a lower limit value of the wet gaseous chlorine dioxide concentration; and a circulation fan air flow rate adjusting button 7d which is able to adjust the concentration of the wet gaseous chlorine dioxide by adjusting the air flow rate of the circulation fan installed inside of a sterilization chamber.

The display portion may include a door state display unit 7e which is able to display an opening and closing state of the door of the sterilization apparatus; a communication state display unit 7f which is able to display a communication connection state between the operation panel 7 and the electronic control part 5; a medicine remainder display unit 7g which is provided to display the remainder of the medicine (a stabilized chlorine dioxide); an air flow rate display unit 7h which is able to display the air flow rate of the circulation fan inside the sterilization chamber; a wet gaseous chlorine dioxide state display unit 7i which is able to display the temperature, humidity and concentration of the wet gaseous chlorine dioxide; a sterilization time display unit 7j which is provided to display the current time, a sterilization start time and a sterilization completion time; and a graph screen 7k which is configured to display in real time in the form of a graph the temperature, humidity and concentration of the wet gaseous chlorine dioxide based on the driving time of the sterilization apparatus.

Moreover, the screen displayed on the operation panel 7 is formed of a logo screen, an operation screen, a door opening and closing screen, a medicine remainder display screen, a gas removal screen and an operation completion screen, which will be automatically displayed in response to a progress state of a sterilization.

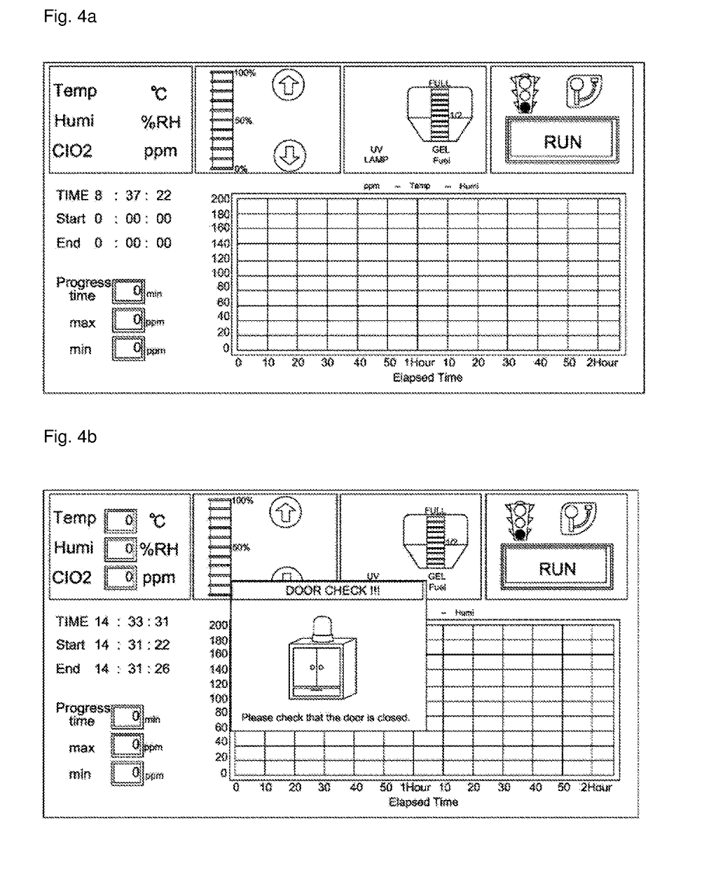

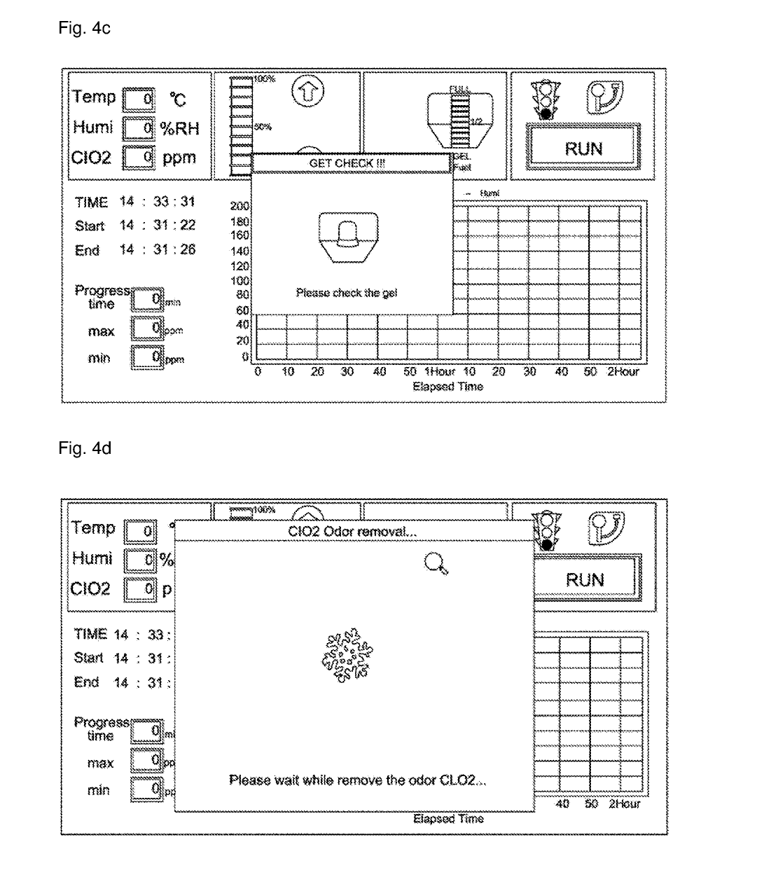

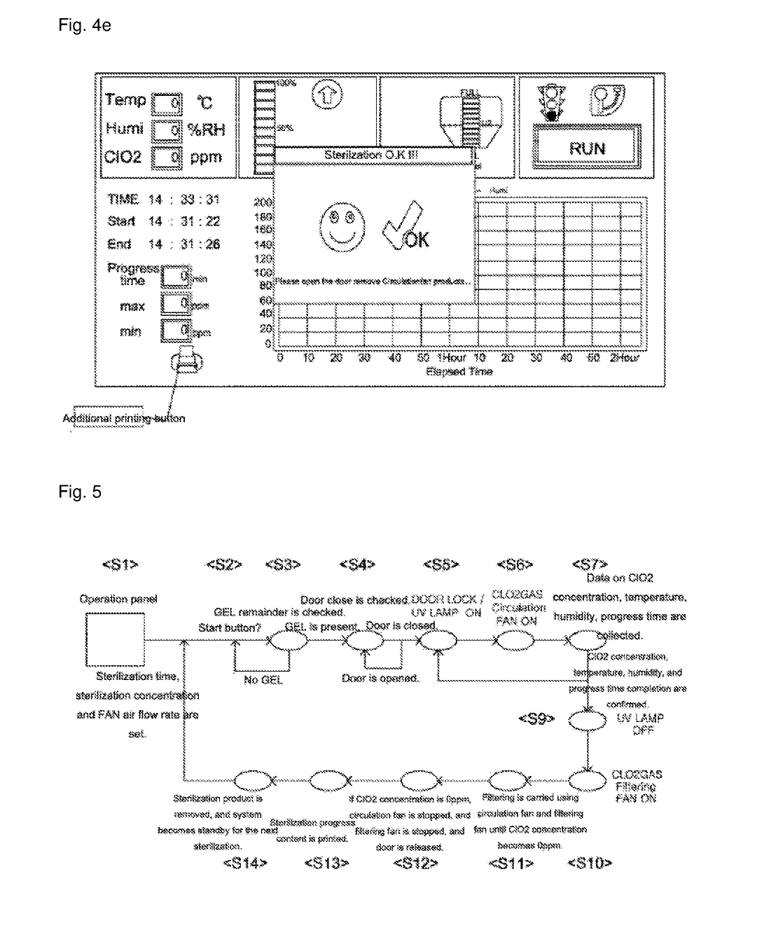

The logo screen (not illustrated) is a screen where a logo randomly designated by a user can be displayed when the sterilization apparatus is turned on, and the operation screen, as illustrated in FIG. 4A, is an initial screen where a sterilization condition can be inputted, and the door opening and closing screen, as illustrated in FIG. 4B, is a screen where a door state check guide message is displayed together with an alarm sound when the user pushes the start button, and the medicine remainder display screen, as illustrated in FIG. 4C, is a screen wherein the medicine remainder guide message is displayed together with an alarm sound after the display of the door opening and closing screen, and the gas removal screen, as illustrated in FIG. 4D, is a screen where a wet gaseous chlorine dioxide removal state guide message id displayed while the wet gaseous chlorine dioxide is being removed in the sterilization chamber, and the operation completion screen, as illustrated in FIG. 4E, is a screen where an operation completion guide message is displayed. When the sterilization object is unloaded by opening the door of the sterilization apparatus, the operation completion screen will disappear. The operation completion screen may include a button to additionally output a result of the sterilization.

The sterilization apparatus using a wet gaseous chlorine dioxide which is equipped with the above-described configuration has an effect on sterilizing even spores, under an environment of a room temperature and a normal pressure, which are the sterilization targets including an unpacked medical instrument, metallic and polymer medical instruments which are weak to a high temperature and a high pressure, a medical product made of an absorbable porous material, and a medical instrument packed with a Tyvek.

Moreover, the sterilization method using a gaseous chlorine dioxide may include generating a wet gaseous chlorine dioxide having a humidity of 65%˜95% by radiating to a stabilized chlorine dioxide an ultraviolet ray having the maximum peak wavelength of 253.7 nm and a radiation output of 1.2˜11 mW/cm² in a gaseous chlorine dioxide generating part 3; and sterilizing spores in such a way to expose a sterilization object to the wet gaseous chlorine dioxide for a predetermined time.

More specifically, the sterilization method using a wet gaseous chlorine dioxide, as illustrated in FIG. 5, may include a sterilization condition setting step (S1) wherein a sterilization time, a gaseous chlorine dioxide concentration, and a circulation fan air flow rate are set at an operation panel of the sterilization apparatus; an operation selection step (S2) wherein an operation button of an operation/stop button provided at the operation panel 7 is selected; a medicine remainder confirmation step (S3) wherein the remainder of a medicine is confirmed at a medicine remainder display unit; a door closing step (S4) wherein a door is closed after the opening and closing state of the door of the sterilization apparatus is checked; a ultraviolet ray lamp ON step (S5) wherein a ultraviolet ray lamp is turned on as soon as the door is closed and locked; a wet gaseous chlorine dioxide circulation step (S6) wherein the wet gaseous chlorine dioxide is circulated in the sterilization chamber by turning on the circulation fan inside the sterilization chamber; a data collection step (S7) wherein the data on a temperature, humidity, concentration and sterilization progress time of the wet gaseous chlorine dioxide are collected; a confirmation step (S8) wherein the sterilization progress time completion is checked; a ultraviolet ray lamp OFF step (S9) wherein the ultraviolet ray lamp is turned off; a filtering fan ON step (S10) wherein a filtering fan is driven to filter the wet gaseous chlorine dioxide; a wet gaseous chlorine dioxide removal step (S11) wherein the circulation fan and the filtering fan are driven until the concentration of the wet gaseous chlorine dioxide becomes 0 (zero) ppm; a door lock releasing step (S12) wherein when the concentration of the wet gaseous chlorine dioxide is 0 ppm, the circulation fan and the filtering fan are stopped, and the door lock state is released; a sterilization progress content printing step (S13) wherein a sterilization progress content is printed using a printer; and a sterilization standby step (S14) wherein a sterilization product is removed from the sterilization chamber, and a standby state is maintained for the sake of the next sterilization.

The detailed embodiments wherein the sterilization with respect to the sterilization objects is carried out by employing the aid the sterilization apparatus and method which use a wet gaseous chlorine dioxide according to the present invention will be described.

First, a result of the sterilization according to the present invention was measured in such a way that the biological indicator to measure a result of sterilization was a product wherein the product was manufactured by MesaLabs corporation, and the name of the product was Bacillus atrophaeus 9372, and the number of strains was 2.5×10⁶ spore/unit (=2,500,000 spore/unit), and an expiration date was Nov. 14, 2013, and the size thereof was 6 mm×19 mm. A result of the measurement according to the present invention was measured in the above way.

As for the biological indicator, if a TSB culture medium turned yellow after a sterilized carrier was inputted in the TSB culture medium (Trypticase Soy Broth Media) manufactured by MesaLabs corporation and was cultured at 36˜38° C. for 48 hours, it was judged that the bacillus atrophaeus 9372 bacteria was grown, which meant that the sterilization had been failed. If the TSB culture medium maintained an orange color, which was the inherent color thereof, it meant the sterilization was 100% succeeded.

As shown in the photo in FIG. 7, “\textcircled{1} a non-used culture medium” is being sold in an orange color in the TSB culture medium, and if the sterilization of the biological indicator (BI) installed in the sterilization chamber is failed, the color will turn yellow, namely, it will turn like “\textcircled{2} a non-sterilized BI culture medium”, and if the sterilization of the biological indicator (BI) installed in the sterilization chamber is succeeded, the inherent color thereof will maintains an orange color, so it becomes possible to visually confirm like “\textcircled{3} a sterilized BI culture medium”.

The tray was installed in a two-layer structure in the chamber of the wet gaseous chlorine dioxide sterilization apparatus, and the biological indicator (BI) was placed at ten places, for example, a front portion, a rear portion, a left portion, a right portion, a central portion, etc. on the trays of the upper and lower layers, and an experiment was carried out according to the first and tenth embodiments as listed in the table 1 by time and concentration, while changing the concentration of the chlorine dioxide and the types and moisture contents of the stabilized gaseous chlorine dioxides. As a result of the experiments, the sterilization effects of the gaseous chlorine dioxide apparatus for a medical purpose according to the present invention were proved.

TABLE 1

| | Concentration of | Types of stabilized | Content of |
|---|---|---|---|
| Examples of experiments | chlorine dioxide | gaseous chlorine dioxide | moisture |
| Embodiment 1 | 5% (10,000 ppm) | Aqueous solution | 97.5% |
| Embodiment 2 | 4% (40,000 ppm) | Aqueous solution | 96.5% |
| Embodiment 3 | 3% (30,000 ppm) | Aqueous solution | 95.5% |
| Embodiment 4 | 2% (20,000 ppm) | Aqueous solution | 94.5% |
| Embodiment 5 | 1% (10,000 ppm) | Aqueous solution | 93.5% |
| Embodiment 6 | 1% (10,000 ppm) | Semi-solid matter (Gel) | 95.5% |
| Embodiment 7 | 2% (20,000 ppm) | Semi-solid matter (Gel) | 94.5% |
| Embodiment 8 | 3% (30,000 ppm) | Semi-solid matter (Gel) | 93.5% |
| Embodiment 9 | 3% (30,000 ppm) | Semi-solid matter (Gel) | 93.5% |
| Embodiment 10 | 3% (30,000 ppm) | Semi-solid matter (Gel) | 93.5% |
| Comparison example 1 | 3% (30,000 ppm) | Solid matter (Powder) | 4.3% |
| Comparison example 2 | 11.6% (116,000 ppm) | Solid matter (Powder) | 1.1% |

The choline dioxide gas sterilization apparatus used in the embodiments 1 to 10 and the comparison examples 1 and 2 had a sterilization chamber wherein a capacity was 87.36 liters, a height was 400 mm, a width was 520 mm, and a depth was 420 mm. A stabilized chlorine dioxide (an aqueous solution, a semi-solid matter, a solid matter) was inputted in a separation region of a 1-litter capacity formed in the sterilization chamber without any partition wall, and a wet gaseous chlorine dioxide was generated by a photochemical reaction wherein a ultraviolet ray lamp having a radiation output of 1.2˜11 mW/cm² radiated an ultraviolet ray having a wavelength of 253.7 nm.

Moreover, the gaseous chlorine dioxide concentration measuring unit may be a photoionization detecting part sensor which is able to measure the concentration of a gaseous chlorine dioxide in the chamber and has a detection range of 0˜1,000 ppm. The electronic control part is able to efficiently control the concentration of a gaseous chlorine dioxide in the sterilization chamber which is set as a gaseous chlorine dioxide generating part is driven or stops in order for a deviation with respect to a previously set concentration to be maintained within a range of ±10 ppm, while continuously receiving the concentration of the gaseous chlorine dioxide in the sterilization chamber which is measured by the gaseous chlorine dioxide concentration measuring unit. The electronic control part may be formed of an operation panel of a touch screen type which is able to display time, concentration, temperature, humidity and an operation of an internal circulation fan during the whole sterilization procedure, and a printer which is able to output on the form of a printed material.

The stabilized gaseous chlorine dioxide used in the embodiments 1 to 10 was a stabilized chlorine dioxide aqueous solution of 1% (10,000 ppm), 2% (20,000 ppm), 3% (30,000 ppm), 4% (40,000 ppm) and 5% (50,000 ppm) of an undiluted solution which were prepared using a 5% solution of “Anthium Dioxide” prepared by the U.S. international dioxide Inc., and a stabilized chlorine dioxide gel of 1% (10,000 ppm), 2% (20,000 ppm), and 3% (30,000 ppm). The “Anthium Dioxide” 5% solution had more than 94% of a water component. When preparing a semi-solid matter using “Anthium Dioxide” 5% solution, 1˜3% of agar and 0.1˜2% of purified agarose were added as a gelling agent, so the holding moisture of the semi-solid matter was 90˜93%. For this reason, the gaseous chlorine dioxide generated based on the photochemical reaction was a gaseous chlorine dioxide which was wetted in such a way to discharge the moisture held by “Anthium Dioxide aqueous solution” or “Anthium Dioxide semi-solid matter” and was spread in the inside of the chamber.

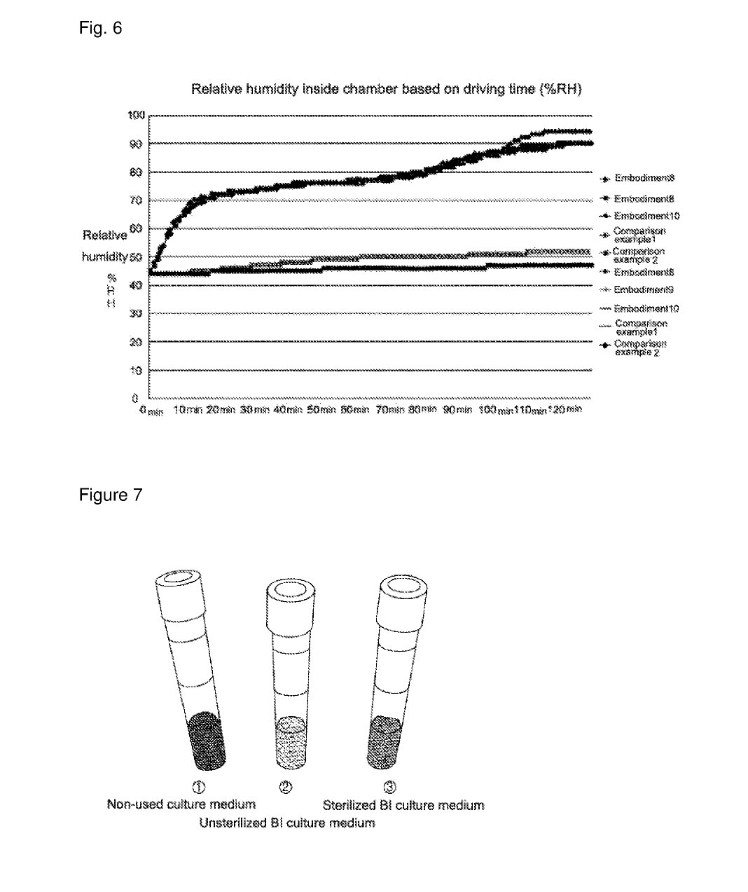

In order to prepare a dry gaseous chlorine dioxide in the comparison example 1, 7 g of Aseptrol tablet prepared by crushing Aseptrol tablet S10-Tab (15% ClO₂ contained) manufactured by Germany Engelhard Corporation and 2 g of citric acid solution were mixed and used. FIG. 6 is a graph showing a relative humidity in the chamber based on a driving time in the embodiments 1 to 10 of the experiment examples and the comparison examples 1 and 2.

The embodiments 1, 2, 3, 4 and 5 used a stabilized chlorine dioxide aqueous solution, and the embodiments 6, 7, 8, 9 and 10 used a stabilized chlorine dioxide semi-solid matter. Since both the aqueous solution and semi-solid matter naturally had a moisture of 93.5%˜97.5%, they had the gaseous chlorine dioxide generated by the photochemical reaction and moisture and were discharged in a state of a wet gaseous chlorine dioxide and were evenly spread in the interior of the sterilization chamber, while penetrating into the Tyvek packing membrane of the biological indicator (BI) installed at ten places, for example, a front portion, a rear portion, a left portion, a right portion, a central portion, etc. on the upper and lower trays inside the chamber, thus activating a 2.5×10⁶ number of Bacillus Atrophaeus 9372 spores, which were present on the strip, into a humidity of over 65%, whereby they were completely sterilized by the gaseous chlorine dioxide, which meant that the sterilization was succeeded.

Results of the experiments in the embodiments 1 to 10 are as follows.

The comparison example 1 used as a chlorine dioxide a stabilized chlorine dioxide of the same concentration of 3% (30,000 ppm) as in the embodiments 3, 8, 9 and 10, and the comparison example 2 used a high concentration of 11.6% (116,000 ppm) which was 3.8 times higher than the concentration of the chlorine dioxide as in the embodiments 3, 8, 9 and 10, but the relative humidity inside the chamber were 52% RH and 47% RH, and it was a dry gaseous chlorine dioxide the relative humidity of which was smaller than 65% RH, whereupon spores were not sterilized, so the sterilization was not carried out. A result of the experiments of the comparison examples 1 and 2 is as follows.

The sterilization apparatus using a wet chlorine dioxide according to the present invention is able to sterilize virus, bacteria, fungus, spores, etc. in the sterilization object in an easier and quick way under an environment of a room temperature and a normal pressure. The present invention is able to allow to provide an easier and convenient sterilization irrespective of any use condition and the material, temperature, pressure, etc. of the sterilization as compared to a conventional gas sterilization and disinfection apparatus which uses a sterilization gas.

The preferred embodiments of the present invention have been described, but it should be understood that the present invention can include all sterilization apparatuses and methods which have the equivalents of the present invention.

1. A sterilization apparatus using a wet gaseous chlorine dioxide, comprising:
a chamber (2) which includes a sterilization chamber (1);
a gaseous chlorine dioxide generating part (3) which is able to generate a wet gaseous chlorine dioxide from a stabilized chlorine dioxide;
a photoionization detecting part (4) which is able to measure the concentration of a gaseous chlorine dioxide in the chamber;
an electronic control part (5) which is provided to control a sterilization process;
a gaseous chlorine dioxide adsorption removal part (6) which is able to remove a gaseous chlorine dioxide remaining in the chamber after the sterilization process has been completed;
an operation panel (7) which is formed of an input portion to input a sterilization condition, and a display portion to display a sterilization state; and
a printer (8) which is provided to output a sterilization history.

2. The apparatus of claim 1, wherein the gaseous chlorine dioxide generating part (3) includes a ultraviolet ray lamp (3a) wherein a chlorine dioxide is formed in a melted state, a semi-solid matter state or a solid matter state to generate a wet gaseous chlorine dioxide having a relative humidity of 65%˜95% via a photochemical reaction, and the moisture content thereof is 70%˜98%, and the concentration of the gaseous chlorine dioxide is 0.1%˜15% (1,000 ppm˜150,000 ppm), and the rest is formed of a stabilized gaseous chlorine dioxide formed of a recipient (an agar), and a predetermined amount of the ultraviolet ray is radiated from the ultraviolet ray lamp toward to the stabilized chlorine dioxide, thus generating a wet gaseous chlorine dioxide.

3. The apparatus of claim 2, wherein the ultraviolet ray lamp has the maximum peak wavelength of 253.7 nm, and the ultraviolet ray radiation output thereof has a capacity of 1.2˜11 mW/cm².

4. The apparatus of claim 3, wherein the photoionization detecting part (4) is provided to detect concentration using a photoionization detecting sensor which is able to ionize and capture the electrons of the gaseous chlorine dioxide among the wet gaseous chlorine dioxide generated in the chamber and transmit the detected measurement values to the electronic control part (5).

5. The apparatus of claim 4, wherein the electronic control part (5) is able to convert a sterilization condition set on the operation panel and a concentration measurement value of the wet gaseous chlorine dioxide received from the photoionization detecting sensor into an electrical signal and display the converted electrical signal, and is able to control the concentration of the gaseous chlorine dioxide, and is able to control a door locking device for the door to be opened or closed only when the gaseous chlorine dioxide remaining in the chamber after the sterilization is completed, is completely removed, and is able to display values and graphs generated during the sterilization procedure, on the operation panel, and the sterilization history is printed using the printer (8), wherein the above control operations are automatically carried out by a control of the electronic control part.

6. The apparatus of claim 5, wherein the gaseous chlorine dioxide adsorption removal part (6) includes a filter containing an active carbon with a particle diameter of 0.01˜0.1 μm, a zeolite with a particle diameter of 0.5˜2 mm, and sodium thiosulfate 5H₂O in order for the wet gaseous chlorine dioxide remaining in the chamber after the sterilization process is completed, to be completely removed, and the filter adsorbs and removes the wet gaseous chlorine dioxide in the chamber by circulating it using a filtering fan after the sterilization process is completed.

7. The apparatus of claim 6, wherein the wet gaseous chlorine dioxide sterilization apparatus is able to sterilize, under an environment of a room temperature and a normal pressure, a sterilization object, for example, an unpacked medical instrument, metallic and polymer-made medical instruments, which are weak to a high temperature, a high pressure and humidity, absorbable and porous medical goods, and a medical instrument which is sealingly packed using a Tyvek, thus sterilizing spores.

8. The apparatus of claim 5, wherein the operation panel (7) is a touch screen panel and is formed of an input portion to input a sterilization condition and a display portion to display a sterilization state.

9. The apparatus of claim 8, wherein the input portion comprises:
a start/stop button (7a) which is provided to control the start and stop of the sterilization apparatus;
a sterilization progress time setting unit (7b) which is able to set a sterilization progress time;
a wet gaseous chlorine dioxide concentration setting unit (7c) which is provided to set an upper limit value and a lower limit value of the wet gaseous chlorine dioxide concentration; and
a circulation fan air flow rate adjusting button (7d) which is able to adjust the concentration of the wet gaseous chlorine dioxide by adjusting the air flow rate of the circulation fan installed inside of a sterilization chamber, and
the display portion comprises:
a door state display unit (7e) which is able to display an opening and closing state of the door of the sterilization apparatus;
a communication state display unit (7f) which is able to display a communication connection state between the operation panel (7) and the electronic control part (5);
a medicine remainder display unit (7g) which is provided to display the remainder of the medicine (a stabilized chlorine dioxide);
an air flow rate display unit (7h) which is able to display the air flow rate of the circulation fan inside the sterilization chamber;
a wet gaseous chlorine dioxide state display unit (7i) which is able to display the temperature, humidity and concentration of the wet gaseous chlorine dioxide;
a sterilization time display unit (7j) which is provided to display the current time, a sterilization start time and a sterilization completion time; and
a graph screen (7k) which is configured to display in real time in the form of a graph the temperature, humidity and concentration of the wet gaseous chlorine dioxide based on the driving time of the sterilization apparatus.

10. The apparatus of claim 8, wherein the screen displayed on the operation panel (7) is formed of a logo screen, an operation screen, a door opening and closing screen, a medicine remainder display screen, a gas removal screen and an operation completion screen, which are separately displayed based on a progress state of the sterilization.

11. A sterilization method using a gaseous chlorine dioxide, comprising:
generating a wet gaseous chlorine dioxide having a humidity of 65%˜95% by radiating to a stabilized chlorine dioxide an ultraviolet ray having the maximum peak wavelength of 253.7 nm and a radiation output of 1.2˜11 mW/cm² in a gaseous chlorine dioxide generating part (3); and
sterilizing spores in such a way to expose a sterilization object to the wet gaseous chlorine dioxide for a predetermined time.

12. The method of claim 11, wherein the sterilization method using a wet gaseous chlorine dioxide comprises:
a sterilization condition setting step (S1) wherein a sterilization time, a gaseous chlorine dioxide concentration, and a circulation fan air flow rate are set at an operation panel of the sterilization apparatus;
an operation selection step (S2) wherein an operation button of an operation/stop button provided at the operation panel (7) is selected;
a medicine remainder confirmation step (S3) wherein the remainder of a medicine is confirmed at a medicine remainder display unit;
a door closing step (S4) wherein a door is closed after the opening and closing state of the door of the sterilization apparatus is checked;
a ultraviolet ray lamp ON step (S5) wherein a ultraviolet ray lamp is turned on as soon as the door is closed and locked;
a wet gaseous chlorine dioxide circulation step (S6) wherein the wet gaseous chlorine dioxide is circulated in the sterilization chamber by turning on the circulation fan inside the sterilization chamber;
a data collection step (S7) wherein the data on a temperature, humidity, concentration and sterilization progress time of the wet gaseous chlorine dioxide are collected;
a confirmation step (S8) wherein the sterilization progress time completion is checked;
a ultraviolet ray lamp OFF step (S9) wherein the ultraviolet ray lamp is turned off;
a filtering fan ON step (S10) wherein a filtering fan is driven to filter the wet gaseous chlorine dioxide;
a wet gaseous chlorine dioxide removal step (S11) wherein the circulation fan and the filtering fan are driven until the concentration of the wet gaseous chlorine dioxide becomes 0 (zero) ppm;
a door lock releasing step (S12) wherein when the concentration of the wet gaseous chlorine dioxide is 0 ppm, the circulation fan and the filtering fan are stopped, and the door lock state is released;
a sterilization progress content printing step (S13) wherein a sterilization progress content is printed using a printer; and
a sterilization standby step (S14) wherein a sterilization product is removed from the sterilization chamber, and a standby state is maintained for the sake of the next sterilization.
